(19) United States
(12) Patent Application Publication
(10) Pub. No.: US 2004/0110269 A1
(43) Pub. Date: Jun. 10, 2004

(54) Protection against mycobacterial infections

(76) Inventors: Richard Vipond, Wiltshire (GB); Helen Shuttleworth, Salisbury Wiltshire (GB); Emma Ambrose, Alberta (CA); Nigel Peter Minton, Wiltshire (GB)

Correspondence Address: Sterne Kessler, Goldstein & Fox, Suite 600, 1100 New York Avenue NW, Washington, DC, 20005-3934, US

(21) Appl. No.: 10/432,934

(22) PCT Filed: Nov. 28, 2001
(86) PCT No.: PCT/GB01/05250

(30) Foreign Application Priority Data
Nov. 28, 2000 (GB) 0028966.0

(51) Int. Cl. C12N 1/20

(52) U.S. Cl. 435/252.3

(57) ABSTRACT

The present invention relates to a method of identifying mycobacterial genes which were induced or up-regulated during M. tuberculosis virulence, and to isolated peptide products of said genes. Also provided, are inhibitors of said genes, and antibodies which bind to said peptide products. Further embodiments include DNA and RNA vectors encoding said products, attenuated mycobacteria in which the activity of at least one of said genes or peptide products has been modified, vaccines against mycobacterial infections, and methods of detecting a mycobacterial infection.

The present invention relates to a method of identifying mycobacterial genes which are induced or up-regulated during mycobacterial virulence, to isolated peptide products of said genes, to inhibitors of said genes, to antibodies which bind to said peptide products, to DNA and RNA vectors encoding said products, to attenuated mycobacteria in which the activity of at least one of said genes or peptide products has been modified, to vaccines against mycobacterial infections, and to methods of detecting a mycobacterial infection.

Many microorganisms are capable of forming intracellular infections. These include: infections caused by species of Salmonella, Yersinia, Shigella, Campylobacter, Chlamydia and Mycobacteria. Some of these infections are exclusively intracellular, others contain both intracellular and extracellular components. However, it is the intracellular survival cycle of bacterial infection which is suspected as a main supportive factor for disease progression.

Generally, these microorganisms do not circulate freely in the body, for example, in the bloodstream, and are often not amenable to drug treatment regimes. Where drugs are available, this problem has been exacerbated by the development of multiple drug resistant microorganisms.

A number of factors have contributed to the problem of microbial drug resistance. One is the accumulation of mutations over time and the subsequent horizontal and vertical transfer of the mutated genes to other organisms. Thus, for a given pathogen, entire classes of antibiotics have been rendered inactive. A further factor has been the absence of a new class of antibiotics in recent years. The emergence of multiple drug-resistant pathogenic bacteria represents a serious threat to public health and new forms of therapy are urgently required.

For similar reasons, vaccine therapies have not proved effective against such intracellular microorganisms. Also, increased systemic concentration of antibiotics to improve bioavailability within cells may result in severe side effects.

Mycobacterium tuberculosis and closely related species make up a small group of mycobacteria known as the Mycobacterium tuberculosis complex (MTC). This group comprises four species M. tuberculosis, M. microti, M. bovis and M. africanum which are the causative agent in the majority of tuberculosis (TB) cases throughout the world.

M. tuberculosis is responsible for more than three million deaths a year world-wide. Other mycobacteria are also pathogenic in man and animals, for example M. avium subsp. paratuberculosis which causes Johne's disease in ruminants, M. bovis which causes tuberculosis in cattle, M. abvium and M. intracellulare which cause tuberculosis in immunocompromised patients (eg. AIDS patients, and bone marrow transplant patients) and M. leprae which causes leprosy in humans.

M. tuberculosis infects macrophage cells within the body, particularly alveolar macrophages in the lung. Soon after macrophage infection, most M. tuberculosis bacteria enter, persist and replicate within cellular phagosome vesicles, where the bacteria are sequestered from host defences and extracellular factors.

It is the intracellular survival and multiplication or replication of bacterial infection which is suspected as a main supportive factor for mycobacterial disease progression.

A number of drug therapy regimens have been proposed for combatting M. tuberculosis infections, and currently combination therapy including the drug isoniazid has proved most effective. However, one problem with such treatment regimes is that they are long-term, and failure to complete such treatment can promote the development of multiple drug resistant microorganisms.

A further problem is that of providing an adequate bioavailability of the drug within the cells to be treated. Whilst it is possible to increase the systemic concentration of a drug (eg. by administering a higher dosage) this may result in severe side effects caused by the increased drug concentration.

The effectiveness of vaccine prevention against M. tuberculosis has varied widely. The current M. tuberculosis vaccine, BCG, is an attenuated strain of M. bovis. It is ,effective against severe complications of TB in children, but it varies greatly in its effectiveness in adults particularly across ethnic groups. BCG vaccination has been used to prevent tuberculous meningitis and helps prevent the spread of M. tuberculosis to extra-pulmonary sites, but does not prevent infection. Furthermore, it can not be administered to immunocompromised individuals, a group particularly susceptible to TB, because of the risk of systemic disease resulting from the vaccine.

The limited efficacy of BCG and the global prevalence of TB has led to an international effort to generate new, more effective vaccines. The current paradigm is that protection will be mediated by the stimulation of a Th1 immune response.

BCG vaccination in man was given orally when originally introduced, but that route was discontinued because of loss of viable BCG during gastric passage and of frequent cervical adenopathy. In experimental animal species, aerosol or intra-tracheal delivery of BCG has been achieved without adverse effects, but has varied in efficacy from superior protection than parenteral inoculation in primates, mice and guinea pigs to no apparent advantage over the subcutaneous route in other studies.

There is therefore a need for an improved and/or alternative vaccine or therapeutic agent for combatting mycobacterial infections.

This need is addressed by the present invention.

According to a first aspect, the present invention provides a method of identifying a mycobacterial nucleic acid promoter sequence which, is induced or up-regulated during mycobacterial virulence, said method comprising:

infecting a macrophage target cell with a Mycobacterium tuberculosis host cell, which host cell contains a nucleic acid construct comprising a putative mycobacterial promoter sequence operably linked to a coding sequence of a reporter gene located down-stream from the promoter;

culturing the macrophage under conditions which support mycobacterial virulence; and

identifying a promoter sequence which is induced or up-regulated during virulence by detecting expression of the reporter sequence.

M. tuberculosis is a high containment pathogen, and use of this microorganism is therefore preferably conducted under Advisory Committee for Dangerous Pathogen (ACDP) 3 conditions.

M. tuberculosis pursues a route of intracellular infection, and there are several differences in the behaviour of M. tuberculosis compared to other pathogens. Thus, nucleic acid expression profiles of other bacteria during infection, even other mycobacteria, do not accurately reflect the series of changes which occur during M. tuberculosis infection.

For example, M. tuberculosis does not use type III secretion systems to inject effector molecules to alter host cell behaviour as do Salmonella species nor does it utilise pore forming toxins such as listeriolysin to escape the phagosomal vesicle as found in L. monocytogenes infection.

Pathogenic mycobacteria appear to rely upon a repertoire of gene products which are tightly regulated in response to the interaction with a host cell, many of which have yet to be identified.

In comparison with other mycobacterial pathogens, species such as M. leprae and M. marinum appear to have a thermotropism where the infection is manifested in the periphery of the body where temperatures are lower than the typical 37° C. core body temperature. M. leprae also targets the myelin sheath of the nerve cells and the disease pathology reflects this tissue tropism. M. marinum causes a limited infection resulting in a surface lesion on the skin which can be self-limiting. Other pathogens such as M. avium and M. intracellulare typically cause infections in immunocompromised individuals and do not usually have the ability to cause disease in healthy subjects. M. bovis infection is usually acquired through ingestion of contaminated milk products resulting in infection via the lymph nodes and leading to lymphoadenopathy. Following infection, M. tuberculosis may enter a dormant state leading to a latent infection. This latent infection may persist for decades before reactivation occurs leading to progressive active disease.

Other mycobacterial species may share in common with M. tuberculosis some components of conserved virulence mechanisms. However, with the publication of the genome sequences for M. tuberculosis and M. leprae, and the publication of other partially completed genome sequences (eg. M. smegmatis, and M. bovis), it is clear there are significant differences between the different mycobacterial pathogens that ultimately define their success as pathogens and the characteristics of the pathology of the diseases that they cause.

Thus, M. tuberculosis is a specialised bacterial pathogen typically spread by inhalation. M. tuberculosis has a complex interaction with its host, from initial contact with host macrophage onwards, which it manipulates to its own advantage leading to either progressive active disease, or a persistent latent infection or most likely a limited infection which is cleared or contained indefinitely by the host.

Mycobacterial virulence includes one or more of the events associated with infection by a mycobacterium of its natural target cell. For example, a natural target cell of M. tuberculosis is a macrophage, and virulence with respect to this bacterium and target cell would include any event involved during the sequence of events comprising phagocytosis by the macrophage or uptake via alternative pathways of the bacterium, formation of the phagosome, multiplication of the bacterium within the phagosome, lysis of the phagosome/host cell, and spread of the lesion to a secondary site (ie. haematogenous spread). Thus, virulence conditions are culture conditions which are conducive for a mycobacterium to express at least one gene which would be normally expressed in vivo during infection of the mycobacterium's natural target cell.

In one embodiment, the putative promoter sequence is selected from the species M. phlei, M. smegmatis, M. africanum, M. caneti, M. fortuitum, M. marinum, M. ulcerans, M. tuberculosis, M. bovis, M. microti, M. avium, M. paratuberculosis, M. leprae, M. lepraemurium, M. intracellulare, M. scrofulaceum, M. xenopi, M. genavense, M. kansasii, M. simiae, M. szulgai, M. haemophilum, M. asiaticum, M. malmoense, M. vaccae and M. shimoidei. Of particular interest are members of the MTC, preferably M. tuberculosis or M. bovis.

In another embodiment, the reporter gene is a silent marker of gene expression, and does not require a selection pressure to be applied during the detection step. This is in contrast to other markers, for example, antibiotic resistance markers which require the presence of the antibiotic to permit selection of desirable transformants. The presence of a selection pressure may be undesirable as this pressure may itself induce or cause up-regulation of other genes, or result in the loss of transiently-expressed promoters. The induction or up-regulation of other genes, eg. growth-related genes, may result in increased growth of background mycobacteria and may cause problems in the identification of desirable transformants free of false positives.

The reporter sequences employed in the method of the present invention do not possess their own promoters, and are therefore reliant on the activity of the putative promoter sequence to effect expression thereof. Thus, by culturing mycobacterial host cells under virulence conditions, it is possible to select for those promoters which are active under virulence conditions by detecting expression of the reporter sequence.

The reporter gene is preferably a Green Fluorescent Protein (GFP). Suitable GFPs such as GFPmut 1, 2 or 3 are described in [Cormack et al. (1996) Gene, 173, pp.33-38]. In a preferred embodiment, the GFP is GFPmut2.

In those embodiments in which the reporter sequence encodes a fluorescent protein, the desirable mycobacterial host cell transformants are capable of fluorescence by expression of the reporter sequence.

Thus, in a preferred embodiment, promoters active during virulence are detected by differential fluorescence induction methods (DFI) as, for example, described in Valdivia, R. H. and Falkow, S. (1997) [Science, vol. 277, Sep. 26, 1997, pp. 2007-2011], and desirable transformed host cells containing said promoters may be isolated by fluorescence activated cell sorting (FACS).

In use, the mycobacterial host cell infects a macrophage in vitro. Thereafter, when recovering the mycobacteria (eg. for FACS analysis), phagosomal vesicles containing the bacteria may be recovered and the vesicles analysed (ie. sorted) whole. Alternatively, both the macrophage and phagosomal vesicles contained within are disrupted and the released bacteria are analysed by FACS.

Suitable macrophage cells include human acute-monocytic leukemic cell lines of macrophage lineage: CD14⁺, CD15⁺ (THP-1), murine bone marrow-derived macrophage cell lines, and murine BALB/c tumour derived macrophage-monocyte cell lines (eg. ATCC TIB-67, and ATCC TIB-71).

Example 3 of the present application describes one preferred macrophage assay of the present application. However, any one of numerous conventional macrophage infection assays may be employed in the present invention.

Suitable media for culturing mycobacteria under non-virulence conditions are described in Wayne, L. G. (1994) [in Tuberculosis: Pathogenesis, Protection, and Control published by the American Society for Microbiology, pp. 73-83]. These include Middlebrook 7H9 Medium [see Barker, L. P., et al. (1998) Molec. Microbiol., vol. 29(5), pp. 1167-1177, and WO00/52139 in the name of the present Applicant].

In use, an induced or up-regulated promoter is identified by detecting increased expression of the reporter sequence during macrophage infection when compared to expression of the reporter sequence under culture conditions which do not promote mycobacterial virulence. This minimises the risk of identifying mycobacterial host cells containing putative promoters which are constitutively expressed.

Suitable culture conditions which do not promote mycobacterial virulence (ie. conditions which support non-virulence) include mycobacterial culture conditions which are substantially free from macrophage.

In one embodiment, the non-virulence culture conditions are substantially non-limiting in terms of aerobic growth (eg. pH, temperature, and available nutrients) of the mycobacterial host cell culture. Thus, the mycobacterial host cell culture is preferably cultured under conventional conditions which permit a doubling time of 18-26 hours.

In use, the mycobacterial host cell culture may be cultured in batch, and is preferably harvested during mid- to late-exponential phase. Alternatively, this point in the growth phase may be mimicked under continuous culture conditions employing a steady state growth rate approximating μₘₐₓ which provides a generation time of approximately 18-24 hours.

The preferred culture method employed by the present invention is that of batch fermenter culture. This method permits careful monitoring of growth culture parameters such as pH, temperature, available nutrients, and dissolved oxygen tension (DOT). In particular, temperature and DOT may be strictly controlled.

The identification of transformed host cells containing promoters which are constitutively expressed may be performed before or after identification of promoters expressed during virulence conditions. In a preferred embodiment, constitutively expressed promoters are identified after identification of induced or up-regulated promoters expressed during virulence.

Increased expression means that the signal detected as a result of expression of the reporter sequence when the mycobacterial host cell is cultured under virulence conditions is at least 1.3 times greater than the signal detected when the host cell is cultured under non-virulence conditions. In one embodiment, the signal is at least 2 times, preferably at least 4 times greater when the host cell is cultured under virulence conditions. In another embodiment, the signal is at least 10, preferably at least 20, and particularly preferably at least 30 times greater when the host cell is cultured under virulence conditions.

Naturally, the expression profile for a given gene in a mycobacterial host cell may vary during the course of infection. For example, a gene may be induced or up-regulated most strongly during the early stages of an infection. Thus, the term “increased expression” refers to the maximum signal obtained for a given reporter gene under virulence conditions vis-a-vis non-virulence conditions.

In one embodiment, the “increased expression” concerns the maximum signal obtained within the first four days after macrophage infection, preferably within the first two days after macrophage infection.

In another embodiment, genes switched on or up-regulated at later stages of infection are of interest, in which case a maximum signal 4 days after macrophage infection is preferred. For example, a maximum signal up to 11-12 days after infection may be preferred. However, due to the invasive and destructive nature of a M. tuberculosis infection on a macrophage, a post-infection period of between 4 and 6 days is particularly preferred.

A number of methods may be employed for obtaining putative mycobacterial promoter sequences for use in the present invention. These include:- generation of a library of putative promoter sequences by use of nucleic acid digestion enzymes (see Example 1); and identifying nucleic acid sequence homology to known virulence or previously implicated virulence sequences in other microorganisms (see Example 2).

In application of the above homology identification method, a single promoter construct (rather than a selection of constructs generated from a library) is prepared, and a promoter-reporter unit constructed. It is then possible to assess promoter activity by direct comparison (eg. by FACS) of mycobacterial host cultured under virulence conditions versus non-virulence conditions. Since only a single promoter-reporter construct is employed, there is no need for a separate screening of the host cells.

The method of the present invention permits identification of promoters which are induced or up-regulated during virulence. Once the nucleic acid sequence and orientation of a desirable promoter sequence has been determined, the exact location can be mapped by use of convention nucleic acid homology search computer software (eg. BLASTN, or BLASTX) performed on published sequences of the mycobacterial species concerned. Accordingly, the gene or operon under the control of the identified promoter may be identified.

Thus, in a second aspect of the present invention, there is provided a method of identifying a mycobacterial gene the expression of which is induced or up-regulated during mycobacterial virulence, said method comprising:

identifying a mycobacterial promoter sequence which is induced or up-regulated during infection of a macrophage by a M. tuberculosis host cell, wherein the host cell contains a nucleic acid construct comprising said promoter sequence operably linked to a coding sequence of a reporter gene located down-stream from the promoter;

aligning by sequence homology the nucleic acid sequence of the promoter with published nucleic acid sequence data for the same mycobacterial species; and

identifying the associated nucleic acid coding sequence under the control of said promoter.

Reference to “gene” throughout this specification embraces open reading frames (ORFs).

According to a third aspect of the present invention there is provided an isolated mycobacterial peptide or a fragment or derivative or variant thereof, wherein the peptide is encoded by a mycobacterial gene the expression of which gene is induced or up-regulated during infection of a macrophage by a M. tuberculosis host cell containing said mycobacterial gene.

The various embodiments described for the first and second aspect of the present invention apply equally for the third and subsequent aspects of the present invention.

The terms “isolated,” “substantially pure,” and “substantially homogenous” are used interchangeably to describe a peptide which has been separated from components which naturally accompany it. A peptide is substantially pure when at least about 60 to 75% of a sample exhibits a single peptide sequence. A substantially pure peptide will typically comprise about 60 to 90% w/w of a protein sample, more usually about 95%, and preferably will be over about 99% pure. Peptide purity or homogeneity may be indicated by, for example, polyacrylamide gel electrophoresis of a protein sample, followed by visualizing a single polypeptide band upon staining the gel. Alternatively, higher resolution may be provided by using, for example, HPLC.

A peptide is considered to be isolated when it is separated from the contaminants which accompany it in its natural state. Thus, a peptide which is chemically synthesized or synthesized in a cellular system different from the cell from which it naturally originates will be substantially free from its naturally associated components.

The present invention provides peptides which may be purified from mycobacteria as well as from other types of cells transformed with recombinant nucleic acids encoding these peptides.

If desirable, the amino acid sequence of the proteins of the present invention may be determined by protein sequencing methods.

The terms “peptide”, “oligopeptide”, “polypeptide”, and “protein” are used interchangeably and do not refer to a specific length of the product. These terms embrace post-translational modifications such as glycosylation, acetylation, and phosphorylation.

The term “fragment” means a peptide having at least five, preferably at least ten, more preferably at least twenty, and most preferably at least thirty-five amino acid residues of the peptide which is the gene product of the induced or up-regulated gene in question. The fragment preferably includes at least one epitope of the gene product in question.

The term “variant” means a peptide or peptide “fragment” having at least seventy, preferably at least eighty, more preferably at least ninety percent amino acid sequence homology with the peptide which is the gene product of the induced or up-regulated peptide in question. An example of a “variant” is a peptide or peptide fragment of an induced/up-regulated gene which contains one or more analogs of an amino acid (eg. an unnatural amino acid), or a substituted linkage. The terms “homology” and “identity are considered synonymous in this specification. In a further embodiment, a “variant” may be a mimic of the peptide or peptide fragment, which mimic reproduces at least one epitope of the peptide or peptide fragment. The mimic may be, for example, a nucleic acid mimic, preferably a DNA mimic.

For sequence comparison, typically one sequence acts as a reference sequence, to which test sequences may be then compared. When using a sequence comparison algorithm, test and reference sequences are input into a computer, subsequent coordinates are designated, if necessary, and sequence algorithm program parameters are designated. The sequence comparison algorithm then calculates the percentage sequence identity for the test sequence(s) relative to the reference sequence, based on the designated program parameters.

Optimal alignment of sequences for comparison may be conducted, for example, by the local homology alignment algorithm of Smith and Waterman [Adv. Appl. Math. 2: 484 (1981)], by the algorithm of Needleman & Wunsch [J. Mol. Biol. 48: 443 (1970)] by the search for similarity method of Pearson & Lipman [Proc. Nat'l. Acad. Sci. USA 85: 2444 (1988)], by computer implementations of these algorithms (GAP, BESTFIT, FASTA, and TFASTA—Sequence Analysis Software Package of the Genetics Computer Group, University of Wisconsin Biotechnology Center, 1710 University Avenue, Madison, Wis. 53705), or by visual inspection [see Current Protocols in Molecular Biology, F. M. Ausbel et al, eds, Current Protocols, a joint venture between Greene Publishing Associates, In. And John Wiley & Sons, Inc. (1995 Supplement) Ausbubel].

Examples of algorithms suitable for determining percent sequence similarity are the BLAST and BLAST 2.0 algorithms [see Altschul (1990) J. Mol. Biol. 215: pp. 403-410; and “http://www.ncbi.nim.nih.gov/” of the National Center for Biotechnology Information].

In a preferred homology comparison, the identity exists over a region of the sequences that is at least 10 amino acids, preferably at least 20 amino acids, more preferably at least 35 amino acids in length.

The term “derivative” means a protein comprising the peptide (or fragment, or variant thereof) which peptide is the gene product of the induced or up-regulated gene in question. Thus, a derivative may include the peptide in question, and a further peptide sequence which may introduce one or more additional epitopes. The further peptide sequence should preferably not interfere with the basic folding and thus conformational structure of the peptide in question. Examples of a “derivative” are a fusion protein, a conjugate, and a graft. Thus, two or more peptides (or fragments, or variants) may be joined together to form a derivative. Alternatively, a peptide (or fragment, or variant) may be joined to an unrelated molecule (eg. a second, unrelated peptide). Derivatives may be chemically synthesized, but will be typically prepared by recombinant nucleic acid methods. Additional components such as lipid, and/or polysaccharide, and/or polyketide components may be included.

All of the molecules “fragment”, “variant” and “derivative” have a common antigenic cross-reactivity and/or substantially the same in vivo biological activity as the peptide product of the induced or up-regulated gene in question from which they are derived. For example, an antibody capable of binding to a fragment, variant or derivative would be also capable of binding to the gene product of the induced or up-regulated gene in question. It is a preferred feature that the fragment, variant and derivative each possess the active site of the peptide which is the induced or up-regulated peptide in question. Alternatively, all of the above embodiments of a peptide of the present invention share a common ability to induce a “recall response” of a T-lymphocyte which has been previously exposed to an antigenic component of a mycobacterial infection.

In a preferred embodiment, the peptide is selected from the group consisting of SEQ ID NO: 2; 4; 6; 8; 10; 12; 14; 16; 19; 21; 23; 25; 27; 29; 31; 33; 35; 38; 40; 42; 44; 46; 48;-50; 52; 55; 58; 60; 62; 64; 67; 69; 72; 75; 78; 80; 82; 85; 87; 89; 92; 94; 97; 100; 103; 105; 107; 109; 111; 113; 115; and 117.

A fourth aspect of the invention provides an inhibitor of a mycobacterial peptide, wherein the peptide is encoded by a mycobacterial gene the expression of which gene is induced or up-regulated during infection of a macrophage by a M. tuberculosis host cell containing said mycobacterial gene, and wherein the inhibitor is capable of substantially preventing or inhibiting the mycobacterial peptide from exerting its native biological function or effect.

Inhibition of the mycobacterial peptide may be effected at the nucleic acid level (ie. DNA, or RNA), or at the peptide level.

In a further embodiment, the inhibitor may be an antibiotic capable of targeting the induced or up-regulated mycobacterial gene, or the peptide product thereof. The antibiotic is preferably specific for the gene and/or peptide product.

Inhibitors of the present invention may be prepared utilizing the sequence information provided herein. For example, this may be performed by overexpressing the peptide, purifying the peptide, and then performing X-ray compounds are created which have similar molecular structures to all or portions of the peptide or its substrate. The compounds may be then combined with the peptide and attached thereto so as to block one or more of its biological activities.

Also included within the invention are isolated or recombinant polynucleotides that bind to the regions of the mycobacterial chromosome (eg. promoter, or coding region), or transcription products thereof, containing sequences that are associated with induction/up-regulation of a mycobacterial gene during macrophage infection by M. tuberculosis, including antisense and triplex forming polynucleotides. As used herein, the term “binding” refers to an interaction or complexation between an oligonucleotide and a target nucleotide sequence, mediated through hydrogen bonding or other molecular forces. The term “binding” more specifically refers to two types of internucleotide binding mediated through base-base hydrogen bonding. The first type of binding is “Watson-Crick-type” binding interactions in which adenine-thymine (or adenine-uracil) and guanine-cytosine base-pairs are formed through hydrogen bonding between the bases. An example of this type of binding is the binding traditionally associated with the DNA double helix and in RNA-DNA hybrids; this type of binding is normally detected by hybridization procedures.

A second type of binding is “triplex binding”. In general, triplex binding refers to any type of base-base hydrogen bonding of a third polynucleotide strand with a duplex DNA (or DNA-RNA hybrid) that is already paired in a Watson-Crick manner.

In a preferred embodiment, the inhibitor may be an antisense nucleic acid sequence which is complementary to at least part of the inducible or up-regulatable gene.

The inhibitor, when in the form of a nucleic acid sequence, comprises, in use, at least 15 nucleotides, preferably at least 20 nucleotides, more preferably at least 30 nucleotides, and most preferably at least 50 nucleotides.

In a fifth aspect there is provided an antibody which binds to a peptide encoded by a gene, or to a fragment or variant or derivative of said peptide, the expression of which gene is induced or up-regulated during infection of a macrophage by a M. tuberculosis host cell containing said gene,

The antibody preferably has specificity for the peptide in question, and following binding thereto may initiate coating of a mycobacterium expressing said peptide. Coating of the bacterium preferably leads to opsonization thereof. This, in turn, leads to the bacterium being destroyed. It is preferred that the antibody is specific for the mycobacterium (eg. species and/or strain) which is to be targeted.

Opsonization by antibodies may influence cellular entry and spread of mycobacteria in phagocytic and non-phagocytic cells by preventing or modulating receptor-mediated entry and replication in macrophages.

The peptides, fragments, variants or derivatives of the present invention may be used to produce antibodies, including polyclonal and monoclonal. If polyclonal antibodies are desired, a selected mammal (eg. mouse, rabbit, goat, horse, etc.) is immunized with an immunogenic polypeptide. Serum from the immunized animal is collected and treated according to known procedures. If serum containing polyclonal antibodies to a desired mycobacterial epitope contains antibodies to other antigens, the polyclonal antibodies may be purified by immunoaffinity chromatography.

Alternatively, general methodology for making monoclonal antibodies by hybridomas involving, for example, preparation of immortal antibody-producing cell lines by cell fusion, or other techniques such as direct transformation of B lymphocytes with oncogenic DNA, or transfection with Epstein-Barr virus may be employed.

The antibody employed in this aspect of the invention may belong to any antibody isotype family, or may be a derivative or mimic thereof. Reference to antibody throughout this specification embraces recombinantly produced antibody, and any part of an antibody which is capable of binding to a mycobacterial antigen.

In one embodiment the antibody belongs to the IgG, IgM or IgA isotype families.

In a preferred embodiment, the antibody belongs to the IgA isotype family. Reference to the IgA isotype throughout this specification includes the secretory form of this antibody (ie. sigA). The secretory component (SC) of sigA may be added in vitro or in vivo. In the latter case, the use of a patient's natural SC labelling machinery may be employed.

In one embodiment, the antibody may be raised against a peptide from a member of the MTC, preferably against M. tuberculosis.

In a preferred embodiment, the antibody is capable of binding to a peptide selected from the group consisting of SEQ ID NO: 2; 4; 6; 8; 10; 12; 14; 16; 19; 21; 23; 25; 27; 29; 31; 33; 35; 38; 40; 42; 44; 46; 48; 50; 52; 55; 58; 60; 62; 64; 67; 69; 72; 75; 78; 80; 82; 85; 87; 89; 92; 94; 97; 100; 103; 105; 107; 109; 111; 113; 115; and 117 (or fragment, variant, of derivative thereof). The antibodies of the present invention are preferably employed in an isolated form.

In a further embodiment, the antigen is an exposed component of a mycobacterial bacillus. In another embodiment, the antigen is a cell surface component of a mycobacterial bacillus.

The antibody of the present invention may be polyclonal, but is preferably monoclonal.

Without being bound by any theory, it is possible that following mycobacterial infection of a macrophage, the macrophage is killed and the bacilli are released. It is at this stage that the mycobacteria are considered to be most vulnerable to antibody attack. Thus, it is possible that the antibodies of the present invention act on released bacilli following macrophage death, and thereby exert a post-infection effect.

It is possible that the passive protection aspect (ie. delivery of antibodies) of the present invention is facilitated by enhanced accessibility of the antibodies of the present invention to antigens on mycobacterial bacilli. It is possible that antibody binding may block macrophage infection by steric hindrance or disruption of its oligomeric structure. Thus, antibodies acting on mycobacterial bacilli released from killed, infected macrophages may interfere with the spread of re-infection to fresh macrophages. This hypothesis involves a synergistic action between antibodies and cytotoxic T cells, acting early after infection, eg. γ\overline{o} and NK T cells, but could later involve also CD8 and CD4 cytotoxic T cells.

According to a sixth aspect of the invention there is provided an attenuated mycobacterium in which a gene has been modified, the expression of which gene is induced or up-regulated during infection of a macrophage by a M. tuberculosis host cell containing said mycobacterial gene, thereby rendering the mycobacterium substantially non-pathogenic.

The term “modified” refers to any genetic manipulation such as a nucleic acid or nucleic acid sequence replacement, a deletion, or an insertion which renders the mycobacterium substantially non-pathogenic or substantially incapable of macrophage infection. In one embodiment the entire inducible or up-regulatable gene may be deleted.

In one embodiment, the modification may be effected by a nucleic acid sequence encoding an anti-sense nucleic acid sequence to the induced or up-regulated gene, or a transcription product thereof. Thus, by including such a nucleic acid sequence in a mycobacterium, for example in the form of a plasmid, expression of the peptide product of the induced or up-regulated gene may be reduced or substantially inhibited.

In a preferred embodiment, the gene to be modified has a wild-type coding sequence corresponding to one of the group consisting of SEQ ID NO: 3; 5; 7; 9; 11; 13; 15; 17; 20; 22; 24; 26; 28; 30; 32; 34; 36; 39; 41; 43; 45; 47; 49; 51; 53; 56; 59; 61; 63; 65; 68; 70; 73; 76; 79; 81; 83; 86; 88; 90; 93; 95; 98; 101; 104; 106; 108; 110; 112; 114; 116; and 118.

It will be appreciated that the wild-type sequences may include minor variations depending on the Database employed. Reference to wild-type simply means that the sequence in question occurs in nature.

A seventh aspect of the present invention provides an attenuated microbial carrier, comprising a peptide encoded by a gene, or a fragment or variant or derivative of said peptide, the expression of which gene is induced or up-regulated during infection of a macrophage by a M. tuberculosis host cell containing said gene.

In use, the peptide (or fragment, variant or derivative) is either at least partially exposed at the surface of the carrier, or the carrier becomes degraded in vivo so that at least part of the peptide (or fragment, variant or derivative) is otherwise exposed to a host's immune system.

In a preferred embodiment, the peptide is selected from the group consisting of SEQ ID NO: 2; 4; 6; 8; 10; 12; 14; 16; 19; 21; 23; 25; 27; 29; 31; 33; 35; 38; 40; 42; 44; 46; 48; 50; 52; 55; 58; 60; 62; 64; 67; 69; 72; 75; 78; 80; 82; 85; 87; 89; 92; 94; 97; 100; 103; 105; 107; 109; 111; 113; 115; and 117.

In one embodiment, the attenuated microbial carrier is selected from the group consisting of attenuated salmonella, attenuated vaccinia virus, attenuated fowlpox virus, or attenuated M. bovis (eg. BCG strain).

According to an eighth aspect of the invention there is provided a DNA plasmid comprising a promoter, a polyadenylation signal, and a DNA sequence which corresponds to the coding sequence of a mycobacterial gene, or a fragment or variant or derivative of said DNA sequence, the expression of which gene is induced or up-regulated during infection of a macrophage by a M. tuberculosis host cell containing said mycobacterial gene, wherein the promoter and polyadenylation signal are operably linked to the DNA sequence.

The term DNA fragment” used in this invention will usually comprise at least about 5 codons (15 nucleotides), more usually at least about 7 to 15 codons, and preferably at least about 35 codons. This number of nucleotides is usually about the minimal length required for a successful probe that would hybridize specifically with such a sequence.

In preferred embodiments, the DNA “fragment” has a nucleotide length which is at least 50%, preferably at least 70%, and more preferably at least 80% that of the coding sequence of the corresponding induced/up-regulated gene.

The term DNA “variant” means a DNA sequence which has substantial homology or substantial similarity to the coding sequence (or a fragment “substantially homologous” (or “substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95 to 98% of the nucleotide bases. Homology determination is performed as described supra for peptides.

Alternatively, a DNA “variant” is substantially homologous (or substantially similar) with the coding sequence (or a fragment thereof) of an induced/up-regulated gene when they are capable of hybridizing under selective hybridization conditions. Selectivity of hybridization exists when hybridization occurs which is substantially more selective than total lack of specificity. Typically, selective hybridization will occur when there is at least about 65% homology over a stretch of at least about 14 nucleotides, preferably at least about 70%, more preferably at least about 75%, and most preferably at least about 90%. See, Kanehisa (1984) Nuc. Acids Res. 12:203-213. The length of homology comparison, as described, may be over longer stretches, and in certain embodiments will often be over a stretch of at least about 17 nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration (eg. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. However, the combination of parameters is much more important than the measure of any single parameter. See, eg., Wetmur and Davidson (1968) J. Mol. Biol. 31:349-370.

The term DNA “derivative” means a DNA polynucleotide which comprises a DNA sequence (or a fragment, or variant thereof) corresponding to the coding sequence of the induced/up-regulated gene and an additional DNA sequence which is not naturally associated with the DNA sequence corresponding to the coding sequence. The comments on peptide derivative supra also apply to DNA “derivative”. A “derivative” may, for example, include two or more coding sequences of a mycobacterial operon which is induced during macrophage infection. Thus, depending on the presence or absence of a non-coding region between the coding sequences, the expression product/s of such a “derivative” may be a fusion protein, or separate peptide products encoded by the individual coding regions.

The above terms DNA “fragment”, “variant”, and “derivative” have in common with each other that the resulting peptide products have cross-reactive antigenic properties which are substantially the same as those of the corresponding wild-type peptide. Preferably all of the peptide products of the above DNA molecule embodiments of the present invention bind to an antibody which also binds to the wild-type peptide. Alternatively, all of the above peptide products are capable of inducing a “recall response” of a T lymphocyte which has been previously exposed to an antigenic component of a mycobacterial infection.

The promoter and polyadenylation signal are preferably selected so as to ensure that the gene is expressed in a eukaryotic cell. Strong promoters and polyadenylation signals are preferred.

In a related aspect, the present invention provides an isolated RNA molecule which is encoded by a DNA sequence of the present invention, or a fragment or variant or derivative of said DNA sequence.

An “isolated” RNA is an RNA which is substantially separated from other mycobacterial components that naturally accompany the sequences of interest, eg., ribosomes, polymerases, and other mycobacterial polynucleotides such as DNA and other chromosomal sequences.

The above RNA molecule may be introduced directly into a host cell as, for example, a component of a vaccine.

Alternatively the RNA molecule may be incorporated into an RNA vector prior to administration.

The polynucleotide sequences (DNA and RNA) of the present invention include a nucleic acid sequence which has been removed from its naturally occurring environment, and recombinant or cloned DNA isolates and chemically synthesized analogues or analogues biologically synthesized by heterologous systems.

The term “recombinant” as used herein intends a polynucleotide of genomic, cDNA, semisynthetic, or synthetic origin which, by virtue of its origin or manipulation: (1) is not associated with all or a portion of a polynucleotide with which it is associated in nature; or (2) is linked to a polynucleotide other than that to which it is linked in nature; and (3) does not occur in nature. This artificial combination is often accomplished by either chemical synthesis means, or by the artificial manipulation of isolated segments of nucleic acids, eg., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

In embodiments of the invention the polynucleotides may encode a peptide which is induced or up-regulated during macrophage infection. A nucleic acid is said to “encode” a peptide if, in its native state or when manipulated, it can be transcribed and/or translated to produce the peptide or a fragment or variant or derivative thereof. The anti-sense strand of such a nucleic acid is also said to encode the sequence.

Also contemplated within the invention are expression vectors comprising the polynucleotide of interest. Expression vectors generally are replicable polynucleotide constructs that encode a peptide operably linked to suitable transcriptional and translational regulatory elements. Examples of regulatory elements usually included in expression vectors are promoters, enhancers, ribosomal binding sites, and transcription and translation initiation and termination sequences. These regulatory elements are operably linked to the sequence to be translated. A nucleic acid sequence is operably linked when it is placed into a functional relationship with another nucleic acid sequence. For instance, a promoter is operably linked to a coding sequence if the promoter affects its transcription or expression. Generally, operably linked means that the DNA sequences being linked are contiguous and, where necessary to join two protein coding regions, contiguous and in reading frame. The regulatory elements employed in the expression vectors containing a polynucleotide encoding a virulence factor are functional in the host cell used for expression.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, eg. by the phosphoramidite method or the triester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

DNA constructs prepared for introduction into a prokaryotic or eukaryotic host will typically comprise a replication system recognized by the host, including the intended DNA fragment encoding the desired peptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals from polypeptides secreted from the host cell of choice may also be included where appropriate, thus allowing the protein to cross and/or lodge in cell membranes, and thus attain its functional topology or be secreted from the cell.

An appropriate promoter and other necessary vector sequences will be selected so as to be functional in the host, and may, when appropriate, include those naturally associated with mycobacterial genes. Promoters such as the trp, lac and phage promoters, tRNA promoters and glycolytic enzyme promoters may be used in prokaryotic hosts. Useful yeast promoters include the promoter regions for metallothionein, 3-phosphoglycerate kinase or other glycolytic enzymes such as enolase or glyceraldehyde-3-phosphate dehydrogenase, enzymes responsible for maltose and galactose utilization, and others.

Appropriate non-native mammalian promoters might include the early and late promoters from SV40 or promoters derived from human cytomegalovirus, murine moloney leukemia virus, mouse mammary tumour virus, avian sarcoma viruses, adenovirus II, bovine papilloma virus or polyoma. In addition, the construct may be joined to an amplifiable gene (e.g., DHFR) so that multiple copies of the gene may be made.

While such expression vectors may replicate autonomously, they may less preferably replicate by being inserted into the genome of the host cell.

Expression and cloning vectors will likely contain a selectable marker, a gene encoding a protein necessary for the survival or growth of a host cell transformed with the vector. The presence of this gene ensures the growth of only those host cells which express the inserts. Typical selection genes encode proteins that (a) confer resistance to antibiotics or other toxic substances, e.g. ampicillin, kanamycin, neomycin, methotrexate, etc.; (b) complement auxotrophic deficiencies; or (c) supply critical nutrients not available from complex media, e.g. the gene encoding D-alanine racemase for Bacilli. The choice of appropriate selectable marker will depend on the host cell.

The vectors containing the nucleic acids of interest can be transcribed in vitro and the resulting RNA introduced into the host cell (e.g., by injection), or the vectors can be introduced directly into host cells by methods which vary depending on the type of cellular host, including electroporation; transfection employing calcium chloride, rubidium chloride calcium phosphate, DEAE-dextran, or other substances; microprojectile bombardment; lipofection; infection (where the vector is an infectious agent, such as a retroviral genome). The cells into which have been introduced nucleic acids described above are meant to also include the progeny of such cells.

Large quantities of the nucleic acids and peptides of the present invention may be prepared by expressing the nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, amphibian or avian species, may also be useful for production of the proteins of the present invention. Propagation of mammalian cells in culture is per se well known. Examples of commonly used mammalian host cell lines are VERO and HeLa cells, Chinese hamster ovary (CHO) cells, and WI38, BHK, and COS cell lines, although other cell lines may be appropriate, e.g., to provide higher expression, desirable glycosylation patterns.

Clones are selected by using markers depending on the mode of the vector construction. The marker may be on the same or a different DNA molecule, preferably the same DNA molecule. The transformant may be screened or, preferably, selected by any of the means well known in the art, e.g., by resistance to such antibiotics as ampicillin, tetracycline.

The polynucleotides of the invention may be inserted into the host cell by any means known in the art, including for example, transformation, transduction, and electroporation. As used herein, “recombinant host cells”, “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transfer DNA, and include the progeny of the original cell which has been transformed. It is understood that the progeny of a single parental cell may not necessarily be completely identical in morphology or in genomic or total DNA complement as the original parent, due to natural, accidental, or deliberate mutation. “Transformation”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

In one embodiment, a DNA plasmid or RNA vector may encode a component of the immune system which is specific to an immune response following challenge with a peptide, wherein said peptide is encoded by a mycobacterial gene which is induced or up-regulated during infection of a macrophage by a M. tuberculosis host cell containing said mycobacterial gene.

An example of such a component is an antibody to the peptide product of an induced or up-regulated gene. Thus, in this embodiment, the nucleic acid sequence (eg. DNA plasmid or RNA vector) encodes the antibody in question.

A ninth aspect provides use of a peptide, an inhibitor, an antibody, an attenuated mycobacterium, an attenuated microbial carrier, a DNA sequence corresponding to the coding sequence of an induced or up-regulated gene or a fragment or variant or derivative of said DNA sequence, a DNA plasmid comprising said DNA sequence or said fragment or variant or derivative, an RNA sequence encoded by said DNA sequence or said fragment or variant or derivative, and/or an RNA vector comprising said RNA sequence, according to the present invention, in the manufacture of a medicament for treating or preventing a mycobacterial infection.

The term “treating” includes post-infection therapy and amelioration of a mycobacterial infection.

The term “preventing” includes reducing the severity/intensity of, or initiation of, a mycobacterial infection.

In a related aspect, there is provided a method of treating or preventing a mycobacterial infection, comprising administration of a medicament selected from the group consisting of a peptide, an inhibitor, an antibody, an attenuated mycobacterium, an attenuated microbial carrier, a DNA sequence corresponding to the coding sequence of an induced or up-regulated gene or a fragment or variant or derivative of said DNA sequence, a DNA plasmid comprising said DNA sequence or said fragment or variant or derivative, an RNA sequence encoded by said DNA sequence or said fragment or variant or derivative, and/or an RNA vector comprising said RNA sequence, according to the present invention, to a patient.

The medicament may be administered by conventional routes, eg. intravenous, intraperitoneal, intranasal routes.

The immunogenicity of the epitopes of the peptides of the invention may be enhanced by preparing them in mammalian or yeast systems fused with or assembled with particle-forming proteins such as, for example, that associated with hepatitis B surface antigen. Vaccines may be prepared from one or more immunogenic peptides of the present invention.

Typically, such vaccines are prepared as injectables, either as liquid solutions or suspensions; solid forms suitable for solution in, or suspension in, liquid prior to injection may also be prepared. The preparation may also be emulsified, or the peptide encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(7′-1′-2′-dipalm itoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The vaccines are conventionally administered parenterally, by injection, for example, either subcutaneously or intramuscularly. Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% -95% of active ingredient, preferably 25%-70%.

The peptides may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be prophylactically and/or therapeutically effective. The quantity to be administered, which is generally in the range of 5 micrograms to 250 micrograms of antigen per dose, depends on the subject to be treated, capacity of the subject's immune system to synthesize antibodies, and the degree of protection desired. Precise amounts of active ingredient required to be administered may depend on the judgment of the practitioner and may be peculiar to each subject.

The vaccine may be given in a single dose schedule, or preferably in a multiple dose schedule. A multiple dose schedule is one in which a primary course of vaccination may be with 1-10 separate doses, followed by other doses given, at subsequent time intervals required to maintain and or reenforce the immune response, for example, at 1-4 months for a second dose, and if needed, a subsequent dose(s) after several months. The dosage regimen will also, at least in part, be determined by the need of the individual and be dependent upon the judgment of the practitioner.

In addition, the vaccine containing the immunogenic mycobacterial antigen(s) may be administered in conjunction with other immunoregulatory agents, for example, immune globulins or cytokines, as well as antibiotics.

The outcome of administering antibody-containing compositions may depend on the efficiency of transmission of antibodies to the site of infection. In the case of a mycobacterial respiratory infection (eg. a M. tuberculosis infection), this may be facilitated by efficient transmission of antibodies to the lungs.

In one embodiment the medicament may be administered intranasally (i.n.). This mode of delivery corresponds to the route of delivery of a M. tuberculosis infection and, in the case of antibody delivery, ensures that antibodies are present at the site of infection to combat the bacterium before it becomes intracellular and also during the period when it spreads between cells.

An intranasal composition may be administered in droplet form having approximate diameters in the range of 100-5000 μm, preferably 500-4000 μm, more preferably 1000-3000 μm. Alternatively, in terms of volume, the droplets would be in the approximate range of 0.001-100 μl, preferably 0.1-50 μl, more preferably 1.0-25 μl.

Intranasal administration may be achieved by way of applying nasal droplets or via a nasal spray.

In the case of nasal droplets, the droplets may typically have a diameter of approximately 1000-3000 μm and/or a volume of 1-25 μl.

In the case of a nasal spray, the droplets may typically have a diameter of approximately 100-1000 μm and/or a volume of 0.001-1 μl.

It is possible that, following i.n. delivery of antibodies, their passage to the lungs is facilitated by a reverse flow of mucosal secretions, although mucociliary action in the respiratory tract is thought to take particles within the mucus out of the lungs. The relatively long persistence in the lungs, fast clearance from the bile and lack of transport to the saliva of some antibodies suggest the role of mucosal site specific mechanisms.

In a different embodiment, the medicament may be delivered in an aerosol formulation. The aerosol formulation may take the form of a powder, suspension or solution.

The size of aerosol particles is one factor relevant to the delivery capability of an aerosol. Thus, smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli.

The aerosol particles may be delivered by way of a nebulizer or nasal spray.

In the case of aerosol delivery of the medicament, the particles may have diameters in the approximate range of 0.1-50 μm, preferably 1-25 μm, more preferably 1-5 μm.

The aerosol formulation of the medicament of the present invention may optionally contain a propellant and/or surfactant.

By controlling the size of the droplets which are to be administered to a patient to within the defined range of the present invention, it is possible to avoid/minimise inadvertent antigen delivery to the alveoli and thus avoid alveoli-associated pathological problems such as inflammation and fibrotic scarring of the lungs.

I.n. vaccination engages both T and B cell mediated effector mechanisms in nasal and bronchus associated mucosal tissues, which differ from other mucosae-associated lymphoid tissues.

The protective mechanisms invoked by the intranasal route of administration may include: the activation of T lymphocytes with preferential lung homing; upregulation of co-stimulatory molecules, eg. B7.2; and/or activation of macrophages or secretory IgA antibodies.

Intranasal delivery of antigens may facilitate a mucosal antibody response which is favoured by a shift in the T cell response toward the Th2 phenotype which helps antibody production. A mucosal response is characterised by enhanced IgA production, and a Th2 response is characterised by enhanced IL-4 production.

Intranasal delivery of mycobacterial antigens allows targeting of the antigens to submucosal B cells of the respiratory system. These B cells are the major local IgA-producing cells in mammals and intranasal delivery facilitates a rapid increase in IgA production by these cells against the mycobacterial antigens.

In one embodiment administration of the medicament comprising a mycobacterial antigen stimulates IgA antibody production, and the IgA antibody binds to the mycobacterial antigen. In another embodiment, a mucosal and/or Th2 immune response is stimulated.

In another embodiment monoclonal antibodies, in particular, may be used to raise anti-idiotype antibodies. Anti-idiotype antibodies are immunoglobulins which carry an “internal image” of the antigen of the infectious agent against which protection is desired. These anti-idiotype antibodies may also be useful for treatment, vaccination and/or diagnosis of mycobacterial infections.

According to a tenth aspect, the peptides (including fragments, derivatives, and variants thereof) of the present invention and antibodies to them are useful in immunoassays to detect the presence of antibodies to mycobacteria or the presence of the virulence associated antigens in biological samples.

Design of the immunoassays is subject to a great deal of variation, and many formats are known in the art. The immunoassay may utilize at least one epitope derived from a peptide of the present invention. In one embodiment, the immunoassay uses a combination of such epitopes. These epitopes may be derived from the same or from different bacterial peptides, and may be in separate recombinant or natural peptides, or together in the same recombinant peptides.

An immunoassay may use, for example, a monoclonal antibody directed towards a virulence associated peptide epitope(s), a combination of monoclonal antibodies directed towards epitopes of one mycobacterial antigen, monoclonal antibodies directed towards epitopes of different mycobacterial antigens, polyclonal antibodies directed towards the same antigen, or polyclonal antibodies directed towards different antigens.

Protocols may be based, for example, upon competition, or direct reaction, or sandwich type assays. Protocols may also, for example, use solid supports, or may be by immunoprecipitation. Most assays involve the use of labelled antibody or polypeptide; the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Typically, an immunoassay for an antibody(s) to a peptide, will involve selecting and preparing the test sample suspected of containing the antibodies, such as a biological sample, then incubating it with an antigenic (i.e., epitope-containing) peptide(s) under conditions that allow antigen-antibody complexes to form, and then detecting the formation of such complexes. The immunoassay may be of a standard or competitive type.

The peptide is typically bound to a solid support to facilitate separation of the sample from the peptide after incubation. Examples of solid supports that can be used are nitrocellulose (eg. in membrane or microtiter well form), polyvinyl chloride (eg. in sheets or microtiter wells), polystyrene latex (eg. in beads or microtiter plates, polyvinylidine fluoride (known as Immulon), diazotized paper, nylon membranes, activated beads, and Protein A beads. For example, Dynatech Immulon microtiter plates or 60 mm diameter polystyrene beads (Precision Plastic Ball) may be used. The solid support containing the antigenic peptide is typically washed after separating it from the test sample, and prior to detection of bound antibodies.

Complexes formed comprising antibody (or, in the case of competitive assays, the amount of competing antibody) are detected by any of a number of known techniques, depending on the format. For example, unlabeled antibodies in the complex may be detected using a conjugate of antixenogeneic Ig complexed with a label (eg. an enzyme label).

In immunoassays where the peptides are the analyte, the test sample, typically a biological sample, is incubated with antibodies directed against the peptide under conditions that allow the formation of antigen-antibody complexes. It may be desirable to treat the biological sample to release putative bacterial components prior to testing. Various formats can be employed. For example, a “sandwich assay” may be employed, where antibody bound to a solid support is incubated with the test sample; washed; incubated with a second, labeled antibody to the analyte, and the support is washed again. Analyte is detected by determining if the second antibody is bound to the support. In a competitive format, a test sample is usually incubated with antibody and a labeled, competing antigen is also incubated, either sequentially or simultaneously.

Also included as an embodiment of the invention is an immunoassay kit comprised of one or more peptides of the invention, or one or more antibodies to said peptides, and a buffer, packaged in suitable containers.

As used herein, a “biological sample” refers to a sample of tissue or fluid isolated from an individual, including but not limited to, for example, plasma, serum, spinal fluid, lymph fluid, the external sections of the skin, respiratory, intestinal, and genitourinary tracts, tears, saliva, milk, blood cells, tumours, organs, and also samples of in vitro cell culture constituents (including but not limited to conditioned medium resulting from the growth of cells in cell culture medium, putatively virally infected cells, recombinant cells, and cell components).

In a related diagnostic assay, the present invention provides nucleic acid probes for detecting a mycobacterial infection.

Using the polynucleotides of the present invention as a basis, oligomers of approximately 8 nucleotides or more can be prepared, either by excision from recombinant polynucleotides or synthetically, which hybridize with the mycobacterial sequences, and are useful in identification of mycobacteria.

The probes are a length which allows the detection of the induced or up-regulated sequences by hybridization. While 6-8 nucleotides may be a workable length, sequences of 10-12 nucleotides are preferred, and at least about 20 nucleotides appears optimal. These probes can be prepared using routine methods, including automated oligonucleotide synthetic methods. For use as probes, complete complementarity is desirable, though it may be unnecessary as the length of the fragment is increased.

For use of such probes as diagnostics, the biological sample to be analyzed, such as blood or serum, may be treated, if desired, to extract the nucleic acids contained therein. The resulting nucleic acid from the sample may be subjected to gel electrophoresis or other size separation techniques; alternatively, the nucleic acid sample may be dot blotted without size separation. The probes are usually labeled. Suitable labels, and methods for labeling probes are known in the art, and include, for example, radioactive labels incorporated by nick translation or kinasing, biotin, fluorescent probes, and chemiluminescent probes. The nucleic acids extracted from the sample are then treated with the labeled probe under hybridization conditions of suitable stringencies.

The probes may be made completely complementary to the virulence encoding polynucleotide. Therefore, usually high stringency conditions are desirable in order to prevent false positives. The stringency of hybridization is determined by a number of factors during hybridization and during the washing procedure, including temperature, ionic strength, length of time, and concentration of formamide.

It may be desirable to use amplification techniques in hybridization assays. Such techniques are known in the art and include, for example, the polymerase chain reaction (PCR) technique.

The probes may be packaged into diagnostic kits. Diagnostic kits include the probe DNA, which may be labeled; alternatively, the probe DNA may be unlabeled and the ingredients for labeling may be included in the kit in separate containers. The kit may also contain other suitably packaged reagents and materials needed for the particular hybridization protocol, for example, standards, as well as instructions for conducting the test.

In a preferred embodiment, a peptide (or fragment or variant or derivative) of the present invention is used in a diagnostic assay to detect the presence of a T-lymphocyte, which T-lymphocyte has been previously exposed to an antigenic component of a mycobacterial infection in a patient.

In more detail, a T-lymphocyte which has been previously exposed to a particular antigen will be activated on subsequent challenge by the same antigen. This activation provides a means for identifying a positive diagnosis of mycobacterial infection. In contrast, the same activation is not achieved by a T-lymphocyte which has not been previously exposed to the particular antigen.

The above “activation” of a T-lymphocyte is sometimes referred to as a “recall response” and may be measured, for example, by determining the release of interferon (eg. IFN-γ) from the activated T-lymphocyte. Thus, the presence of a mycobacterial infection in a patient may be determined by the release of a minimum concentration of interferon from a T-lymphocyte after a defined time period following in vitro challenge of the T-lymphocyte with a peptide (or fragment or variant or derivative) of the present invention.

In use, a biological sample containing T-lymphocytes is taken from a patient, and then challenged with a peptide (or fragment, variant, or derivative thereof) of the present invention.

The above T-lymphocyte diagnostic assay may include an antigen presenting cell (APC) expressing at least one major histocompatibility complex (MHC) class II molecule expressed by the patient in question. The APC may be inherently provided in the biological sample, or may be added exogenously. In one embodiment, the T-lymphocyte is a CD4 T-lymphocyte.

### EXAMPLE 1

### Generation of Putative Promoter Library

The routine molecular biology methods employed were as described in the standard reference protocols (Ausubel F. M., Brent R., Kingston R. E., Moore D. D., Seidman J. G., Smith J. A. and Struhl K. (1992). “Current protocols in molecular biology”. John Wiley & Sons, Chichester).

The promoter library was prepared by partial restriction endonuclease digestion (Sau 3A1) of M. tuberculosis H37Rv chromosomal DNA. Digested DNA was size fractionated and fragments of 1-2 kb in size recovered using the Qiaex II Gel Extraction Kit (Qiagen Ltd).

Recovered fragments were ligated into the vector pCREP8GFPTTL which had been linearised with Bgl II and dephosphorylated. The vector PCREP8GFPTTL is based upon the vector pCREP8 provided by Dr P O'Gaora, ICSM, London which is itself based upon the pNG2 replicon (Radford and Hodgson, 1991, Plasmid 25: 149-153).

Modifications of pCREP8 to create pCREP8GFPTTL include replacement of the region encoding Cre recombinase with the gene encoding GFPmut2 (Cormack, Valdivia and Falkow. Gene. 1996, 173: 33-38). In addition transcriptional terminators from CeIA (from C. thermocelum) and Ferredoxin (from C. pasteurianum) genes were inserted upstream and downstream of the GFPmut2 gene respectively. Finally translational stops were introduced in all three reading frames by insertion of a linker between the Bgl II cloning site and the ribosome binding site immediately preceding the ATG start codon of the GFPmut2 gene.

The ligation mixture was transformed into E. coli and resulting transformants banked into 96 well plates to give 23,040 individual clones. Large scale plasmid isolation was performed on pools of 480 clones using Qiagen Maxi plasmid preps (Qiagen Ltd).

### EXAMPLE 2

### Putative Promoters Identified by Sequence Homology

Promoter regions for targeted fusions were selected by screening the annotated M. tuberculosis genome (http://www.sanger.ac.uk/Projects/M_tuberculosis/ or http://pedant.mips.biochem.mpg.de/) for secreted proteins, surface exposed proteins and those with homology to known virulence factors or implicated previously in mycobacterial virulence using text searches.

In this way a number of candidates for targeted fusions may be isolated. Having identified a promoter, the organisation of the surrounding gene structure was examined and, where possible, a promoter was identified.

Mycobacterial promoters lack typical −35 and −10 sequences [Das Gupta S K, Bashyam M D and Tyagi A K (1993). J. Bacteriol., 175: 5186-5192].

In this instance the region immediately upstream of the gene or operon of interest was amplified by PCR using tailed primers and cloned directly into the Bgl II site of pCREP8GFPTTL. The GFPmut2 vector was used as described above using macrophage cell line J774. 1, in vitro culture grown in Middlebrook 7H9 supplemented with 10% OADC and 0.25% Triton WR1339 (synthetic mycobacterial culture medium), or DMEM.

Table 1 lists a number of such promoters.

TABLE 1
FACS analysis of Targeted Promoter-GFP
Fusions in Macrophage Infection Assays

| | | Net |
|---|---|---|
| | | change** |
| | | in % |
| | | popu- |
| | Identity/Predicted function | lation > |
| Promoter | Gene arrangement⁺ | 10³ |
| TF m1 | Invasion loci mce1 operon Rv0167-0174 | 3.3 |
| TF h | sigE operon Rv1221-1224 | 21.7 |
| TF m2 | mce2 operon | 1.46 |
| TF t | Rv2462c trigger factor | 4.41 |
| TF2 | mce3 operon Rv1964-1971 | 12.6 |
| TF3 | probable secreted protein Rv1910c | 2.9 |
| TF7 | Weak similarity to pollen antigens Rv1919c | 6 |
| TF10 | Putative exported protein Rv1477-1478 | 40 |
| TF11 | putative exported protease Rv2671/2672/2673 | 5.42 |
| TF13 | proteolytic subunit Rv2461c | 3.7 |
| TF15 | single gene heat shock protein Rv0384c | 11.5 |
| H37Rv | Non-fluorescing control | −0.1 |
| | Control | |

⁺as annotated in the genome sequence (www.sanger.ac.uk/Projects/M_tuberc)
**increase or decrease over DMEM control hi gated population with greater than 10³ fluorescence

### EXAMPLE 3

### Protocol for Macrophage Infection, Harvesting, FACS Analysis and Sorting, and Recovery of M. tuberculosis

Macrophage infection with batch culture samples of M. tuberculosis. Wells were harvested at intervals of 24 hr, 48 hr and 72 hrs post infection.

Materials

Mouse macrophage cell line J774A.1 (obtainable from, for example, the ECACC) in 6 well plates were seeded at 5×10⁵ cells per well.

Tissue culture medium—Dulbecco's Modified Eagle Medium supplemented with 200 mM L-glutamate, 10% foetal bovine serum (gamma-irradiated) and 10 mM HEPES.

Batch culture sample of M. tuberculosis was grown to mid-late exponential log (O.D₆₀₀ 0.6-0,8).

Liquid Middlebrook 7H9 medium (Difco) supplemented with 10% OADC enrichment (Difco) and 0.25% Triton WR1339, or organisms were cultured on solid Middlebrook 7H10 medium (Difco) supplemented with 10% OADC enrichment (Difco) for growth of M. tuberculosis outside of macrophage.

Procedure

1. Macrophage were grown until confluent (4×10⁶ cells per well).

2. Bacterial samples were prepared using the following procedure:

2-3 week old colonies were scraped from 7H10+OADC agar plates and resuspended in 10 mls of 7H9+OADC+Triton WR1339. A universal tube (22 mm diameter×90 mm height) containing 10 mls of 7H9+OADC+Triton WR1339 was inoculated with 500 μl of the bacterial suspension. Bacteria were grown on an orbital shaker (200 rpm) at 37° C. for 7 days

500 μls of bacterial culture was inoculated into a universal tube (22 mm diameter×90 mm height) containing 10 mls of 7H9+OADC+Triton WR1339 1 ml. Bacteria were grown on an orbital shaker (200 rpm) at 37° C. for 5 days (O.D₆₀₀ 0.6-0.8).

Bacterial samples were diluted in DMEM to a cell density of 1×10⁷/ml—a multiplicity of approximately 2:1 (bacteria to cells) or less is preferred.

3. Tissue culture medium was removed from each tissue culture well

4. 1 ml of bacterial suspension was added to each well and incubated at 37° C. for 3 hours in an atmosphere of 5% CO₂ in air.

5. The inoculum suspension and was removed and each well washed 4 times with 1×phosphate buffered saline (PBS) pre-warmed to 37° C.

6. 3 ml fresh pre-warmed DMEM was added to each well.

7. Infected monolayers were incubated at 37° C. in an atmosphere of 5% CO₂ in air, for a period of 1, 2 or 3 days.

8. Wells for each sample were harvested at appropriate time points as follows:

i) Culture medium was removed by aspiration and monolayers washed with pre-warmed PBS to remove any external bacteria.

ii) The PBS buffer was removed and replaced with 1 ml of 0.25% Triton X-100 in water.

iii) The monolayers were Incubated for 25 mins before disruption of the monolayer with a pastette to release bacteria from the macrophage.

iv) Samples were transferred to a FACS tube and examined using a FACS scanner/sorter at Cat III collecting highly fluorescent bacteria (typically greater than 10³ logs of fluorescence).

9. Bacteria were recovered by passing the sorted suspensions through a 0.2 μM filter.

10. Recovered bacteria on the filter membrane were cultured on Middlebrook 7H10+OADC agar plates and grown at 37° C. for 2 weeks.

11. Bacteria were scraped from the filter membrane into 10 mIs of 7H9+ADC medium and grow for 5-7 days (37° C., 200 rpm).

Notes:

i) The macrophage infection assay and sorting were repeated until the desired level of enrichment of fluorescent bacteria was attained

ii) Bacteria carrying M. tuberculosis promoters that were highly active outside of the macrophage environment were removed by culturing bacteria in Middlebrook 7H9+ADC medium then FACS sorting to collect the non-fluorescent bacteria. (This selection procedure was carried out between passage 1 and passage 2 of the repeat macrophage infection rounds).

### EXAMPLE 4

### Alternative Macrophage Assay Procedures

1). Triccas, J. A. et al. (1999). Microbiology 145, 2923-2930.

Procedure

Macrophage cells were seeded at 2×10⁵ per well in 24-well plates and incubated at 37° C. in 5% CO₂ for 7 days. Macrophage monolayers were infected with bacteria at a multiplicity of infection (m.o.i) of 1:1. After 4 hours of infection extracellular bacteria were removed by washing 4× with PBS and incubation continued in 5% CO₂.

After 5-6 days of infection, infected macrophages were washed 3× in PBS, scraped into 1 ml PBS and analysed by FACS. To recover bacteria, sorted macrophages were centrifuged and lysed in water plus 0.1% Tween 80. Recovered bacteria were grown in 7H9 medium for 7 days and macrophage infection and sorting repeated until the desired level of enrichment of macrophage/fluorescent bacteria was attained.

To select clones with enhanced intracellular expression the macrophage infection was performed as above, however the macrophage were then lysed with Tween 80 to release the bacteria prior to FACS sorting.

2). Kremer, L. et al. (1995). Mol Microbiol. 17, 913-922.

Procedure (Macrophage Infection Only)

J774A.1 macrophages were seeded in eight-chamber culture slides and incubated overnight at 37° C. in 5% CO₂. Just prior to infection, the monolayer was washed with RPM I1640. Recombinant BCG was added at a m.o.i. of 1-5 bacteria per macrophage.

After an overnight infection at 37° C. in the presence of 5% CO₂, the cells were washed 3× with RPMI to remove possible extracellular bacteria. The macrophage were then examined using a fluorescence microscope.

3). Dhandayuthapani, S. et al. (1995). Mol. Microbiol. 17, 901-912.

Procedure

Macrophages were seeded in 100 mm tissue-culture petri dishes (5×10⁶). After overnight attachment at 37° C. in the presence of 5% CO₂ the macrophage were infected at a m.o.i of 1 bacteria per macrophage. One hour after infection, the monolayers were washed 3× with prewarmed PBS to remove non-ingested bacteria and the DMEM replaced.

The macrophages were harvested 6 days post-infection by washing with PBS and then scraped from the dish. The macrophages were collected by centrifugation and broken by 20-30 passages through a 23-gauge needle: intact cells and nuclei were pelleted and the supernatant containing bacteria collected. The bacteria were then pelleted, washed with PBS and centrifuged again. The final bacterial pellet was suspended in PBS for FACS analysis.

4). Via, L. E. et al. (1996). J. Bacteriol. 178, 3314-3321.

Procedure

J774 macrophage monolayers were seeded at a density of 7.5×10⁶ cells per 100 mm-diameter tissue culture petri dish. After overnight attachment at 37° C. in the presence of 5% CO₂, macrophages were infected at a m.o.i of 10-20 bacteria per macrophage. One hour after infection any extracellular bacteria were removed by washing 3× with prewarmed PBS, then fresh DMEM+5% FBS added.

The medium was replaced every 3-4 days of culture. The infected macrophages were incubated for 10 mins, 3 days, 7 days and 11 days (FACS analysis was performed on the 10 min, 7 day and 11 day samples) before harvesting. At harvest the monolayers were washed 3× with PBS, scraped from the dish, centrifuged then homogenised in 20 mM HEPES (pH 7.2)−250mM sucrose. For FACS the homogenate was then subjected to three rounds of centrifugation to remove unbroken macrophage and macrophage debris, the final bacterial pellet was suspended in PBS. FACS analysis and sorting were carried out as described above in reference 3.

5). Barker L. P. et al. (1998). Mol. Microbiol. 29, 1167-1177.

Procedure

Macrophages were seeded into tissue culture dishes at a concentration of 2×10⁵ cells/ml total volume. Macrophages were grown to semi-confluency before bacteria were added at a m.o.i. of 1-5 bacteria per macrophage. After 4 hours growth at 37° C. in the presence of 5% CO₂ the medium was removed and fresh DMEM supplemented with amikacin 100 μg/ml was added to kill extracellular organisms.

After 24 hours at 32° C. the concentration of amikacin was reduced to 20 μg/ml. At three days after infection (the point at which most phagocytic vesicles contain only one organism) the macrophages are washed in PBS, scraped from the dish and subjected to a very gentle and controlled lysis that disrupts the cell membrane without compromising the nuclear membrane.

Vesicles containing bacteria are then isolated by centrifugation, the supernatant removed and diluted with PBS for FACS analysis. Fluorescent bacterial clones that were sorted away from non-fluorescent vesicles and vesicles containing non-fluorescent organisms were screened further using a confocal microscope.

Those clones that appeared to fluoresce intracellularly but not on 7H10 agar or in tissue culture media were passaged through macrophage again. After 3-4 days of growth the bacteria were harvested from the macrophage by lysing the macrophage with 0.1% Triton-X in PBS for 5 min, after which the detergent was diluted with 9 volumes of PBS ready for FACS analysis.

### EXAMPLE 5

### Elucidation of Mycobacterial Nucleic Acid Coding Sequences under the Transcriptional Control of Identified Promoters

DFI allows identification of a promoter which is induced or up-regulated during infection of macrophage, a key step in the pathogenesis of tuberculosis.

Once the DNA sequence and orientation of a promoter region has been determined the exact location can be mapped by doing a DNA homology search (e.g BLASTN or BLASTX, see http://www.sanger.ac.uk/) on the published M. tuberculosis H37Rv genome to reveal the gene or operon under control of the identified promoter region.

Knockout mutants of the gene(s) under control of the identified promoter may then be prepared to see if they are essential for virulence. The fact that the genes are expressed or up-regulated during the infection means they may be essential for virulence of the organism (as demonstrated by testing a knockout mutant in a suitable model).

These data help identify a) a potential vaccine candidate which the host immune response can be targeted to; b) a gene which when inactivated sufficiently attenuates the organism that it could be considered suitable as a live vaccine; c) a target for the development of a new antibiotic.

A suitable plasmid DNA vector (Tascon R E et al., 1996 Nat. Med. 2:8 888-892; Huygen K et al., 1996 Nat. Med. 2:8 893-898) containing the DNA sequence corresponding to one or more of the genes in an identified operon may be tested as a DNA vaccine in comparison with pre-existing TB vaccines. Similarly, gene products thereof may be tested as a vaccine in a guinea pig protection model.

### EXAMPLE 6

### Delete One or More of the Genes from M. tuberculosis in Order to Attenuate its Virulence while Retaining Immunogenicity

One or more genes that are identified may be disrupted using allelic exchange. In brief, the gene of interest is cloned with 1-2 kb of flanking DNA either side and is inactivated by deletion of part of the coding region and insertion of an antibiotic resistance marker, such as hygromycin.

The manipulated fragment is then transferred to a suitable suicide vector eg. pPR23 and is transformed into the wild-type parent strain of M. tuberculosis. Mutants are recovered by selecting for antibiotic resistant strains. Genotypic analysis (Southern Blotting with a fragment specific to the gene of interest) is performed on the selected strains to confirm that the gene has been disrupted.

The mutant strain is then studied to determine the effect of the gene disruption on the phenotype. In order to use it as a vaccine candidate it would be necessary to demonstrate attenuated virulence. This can be done using either a guinea pig or mouse model of infection. Animals are infected with the mutant strain and the progression of disease is monitored by determining the bacterial load in different organs, in particular the lung and spleen, at specific time points post infection, typically up to 16 weeks.

Comparison is made to animals infected with the wild-type strain which should have a significantly higher bacterial load in the different organs. Long-term survival studies and histopathology can also be used to assess virulence and pathogenicity.

Once attenuated virulence has been established, protection and immunogenicity studies can be performed to assess the potential of the strain as a vaccine. Suitable references for allelic exchange and preparation of TB mutants are McKinney et al., 2000 and Pelicic et al., 1997, [1, 2].

### EXAMPLE 7

### Select One or More of Our Genes, which Encode Proteins that are Immunogenic, and put them into BCG or an Attenuated Strain of M. tuberculosis to Enhance its Overall Immunogenicity

The gene of interest is amplified from the M. tuberculosis genome by PCR. The amplified product is purified and cloned into a plasmid (pMV306) that integrates site specifically into the mycobacterial genome at the attachment site (attB) for mycobacteriophage L5 [3].

BCG is transformed with the plasmid by electroporation, which involves damaging the cell envelope with high voltage electrical pulses, resulting in uptake of the DNA. The plasmid integrates into the BCG chromosome at the attB site generating stable recombinants. Recombinants are selected and are checked by PCR or Southern blotting to ensure that the gene has been integrated. The recombinant strain is then used for protection studies.

### EXAMPLE 8

### Use Recombinant Carriers such as Attenuated Salmonella and the Vaccinia Virus to Express and Present TB Genes

One of the best examples of this type of approach is the use of Modified Vaccinia virus Ankara (MVA) [4]. The gene of interest is cloned into a vaccinia virus shuttle vector, e.g. pSC11. Baby Hamster Kidney (BHK) cells are then infected with wild-type MVA and are transfected with the recombinant shuttle vector. Recombinant virus is then selected using a suitable selection marker and viral plaques, selected and purified.

Recombinant virus is normally delivered as part of a prime-boost regime where animals are vaccinated initially with a DNA vaccine encoding the TB genes of interest under the control of a constitutive promoter. The immune response is boosted by administering recombinant MVA carrying the genes of interest to the animals at least 2 weeks later.

### EXAMPLE 9

### Sub-Unit Vaccines Containing a Single Peptide/Protein or a Combination of Proteins

To prepare sub-unit vaccines with one or more peptides or proteins it is first of all necessary to obtain a supply of protein or peptide to prepare the vaccine. Up to now, this has mainly been achieved in mycobacterial studies by purifying proteins of interest from TB culture. However, it is becoming more common to clone the gene of interest and produce a recombinant protein.

The coding sequence for the gene of interest is amplified by PCR with restriction sites inserted at the N terminus and C terminus to permit cloning in-frame into a protein expression vector such as pET-15b. The gene is inserted behind an inducible promoter such as lacZ. The vector is then transformed into E. coli which is grown in culture. The recombinant protein is over-expressed and is purified.

One of the common purification methods is to produce a recombinant protein with an N-terminal His-tag. The protein can then be purified on the basis of the affinity of the His-tag for metal ions on a Ni-NTA column after which the His-tag is cleaved. The purified protein is then administered to animals in a suitable adjuvant [5].

### EXAMPLE 10

### Plasmid DNA Vaccines Carrying One or More of the Identified Genes

DNA encoding a specific gene is amplified by PCR, purified and inserted into specialised vectors developed for vaccine development, such as pVAX1. These vectors contain promoter sequences, which direct strong expression of the introduced DNA (encoding candidate antigens) in eukaryotic cells (eg. CMV or SV40 promoters), and polyadenlyation signals (eg. SV40 or bovine growth hormone) to stabilise the mRNA transcript.

The vector is transformed into E. coli and transformants are selected using a marker, such as kanamycin resistance, encoded by the plasmid. The plasmid is then recovered from transformed colonies and is sequenced to check that the gene of interest is present and encoded properly without PCR generated mutations.

Large quantities of the plasmid is then produced in E. coli and the plasmid is recovered and purified using commercially available kits (eg. Qiagen Endofree-plasmid preparation). The vaccine is then administered to animals for example by intramuscular injection in the presence or absence of an adjuvant.

### EXAMPLE 11

### Preparation of DNA Expression Vectors

DNA vaccines consist of a nucleic acid sequence of the present invention cloned into a bacterial plasmid. The plasmid vector pVAX1 is commonly used in the preparation of DNA vaccines. The vector is designed to facilitate high copy number replication in E. coli and high level transient expression of the peptide of interest in most mammalian cells (for details see manufacturers protocol for pVAX1 ( catalog no. V260-20 www.invitrogen.com).

The vector contains the following elements

Human cytomegalovirus immediate-early (CMV) promoter for high-level expression in a variety of mammalian cells

T7 promoter/priming site to allow in vitro transcription in the sense orientation and sequencing through the insert

Bovine growth hormone (BGH) polyadenylation signal for efficient transcription termination and polyadenylation of mRNA

kanamycin resistance gene for selection in E. coli

A multiple cloning site

pUC origin for high-copy number replication and growth in E. coli

BGH reverse priming site to permit sequencing through the insert

Vectors may be prepared by means of standard recombinant techniques which are known in the art, for example Sambrook et al., (1989). Key stages in preparing the vaccine are as follows:

The gene of interest is ligated into pVAX1 via one of the multiple cloning sites

The ligation mixture is then transformed into a competent E. coli strain (eg. TOP10) and LB plates containing 50 μg/ml kanamycin are used to select transformants.

Clones are selected and may be sequenced to confirm the presence and orientation of the gene of interest.

Once the presence of the gene has been verified, the vector can be used to transfect a mammalian cell line to check for protein expression. Methods for transfection are known in the art and include, for example, electroporation, calcium phosphate, and lipofection.

Once peptide expression has been confirmed, large quantities of the vector can be produced and purified from the appropriate cell host, e.g. E. coli.

pVAX1 does not integrate into the host chromosome. All non-essential sequences have been removed to minimise the possibility of integration. When constructing a specific vector, a leader sequence may be included to direct secretion of the encoded protein when expressed inside the eukaryotic cell.

Other examples of vectors that have been used are V1 Jns.tPA and pCMV4 (Lefevre et al., 2000; and Vordermeier et al., 2000).

Expression vectors may be used that integrate into the genome of the host, however, it is more common and more preferable to use a vector that does not integrate. The example provided, pVAX1, does not integrate. Integration would lead to the generation of a genetically modified host which raises other issues.

### EXAMPLE 12

### RNA Vaccine

As discussed on page 15 of U.S. Pat. No. 5,783,386, one approach is to introduce RNA directly into the host.

Thus, the vector construct (Example 11) may be used to generate RNA in vitro and the purified RNA then injected into the host. The RNA would then serve as a template for translation in the host cell. In this embodiment. In this embodiment, integration would not occur.

Another option is to use an infectious agent such as the retroviral genome carrying RNA corresponding to the gene of interest. In this embodiment, integration into the host genome will occur.

Another option is the use of RNA replicon vaccines which can be derived from virus vectors such as Sindbis virus or Semliki Forest virus. These vaccines are self-replicating and self-limiting and may be administered as either RNA or DNA which is then transcribed into RNA replicons in vivo. The vector eventually causes lysis of the transfected cells thereby reducing concerns about integration into the host genome. Protocols for RNA vaccine construction are detailed in Cheng, et al. (2001).

### EXAMPLE 13

### Diagnostic Assays Based on Assessing T Cell Responses

For a diagnostic assay based on assessing T cell responses it would be sufficient to obtain a sample of blood from the patient. Mononuclear cells (monocytes, T and B lymphocytes) can be separated from the blood using density gradients such as Ficoll gradients.

Both monocytes and B-lymphocytes are both able to present antigen, although less efficiently than professional antigen presenting cells (APCs) such as dendritic cells. The latter are more localised in lymphoid tissue.

The simplest approach would be to add antigen to the separated mononuclear cells and incubate for a week and then assess the amount of proliferation. If the individual had been exposed to the antigen previously through infection, then T-cell closes specific to the antigen should be more prevalent in the sample and should respond.

It is also possible to separate the different cellular populations should it be desired to control the ratio of T cells to APC's.

Another variation of this type of assay is to measure cytokine production by the responding lymphocytes as a measure of response. The ELISPOT assay described below in Example 14 is a suitable example of this variation.

### EXAMPLE 14

### Detection of Latent Mycobacteria

A major problem for the control of tuberculosis is the presence of a large reservoir of asymptomatic individuals infected with tubercle bacilli. Dormant bacilli are more resistant to front-line drugs.

The presence of latent mycobacteria-associated antigen may be detected indirectly either by detecting antigen specific antibody or T-cells in blood samples.

The following method is based on the method described in Lalvani et al. (2001) in which a secreted antigen, ESAT-6, was identified as being expressed by members of the M. tuberculosis complex but is absent from M. bovis BCG vaccine strains and most environmental mycobacteria. 60-80% of patients also have a strong cellular immune response to ESAT-6. An ex-vivo ELISPOT assay was used to detect ESAT-6 specific T cells.

As applied to the present invention:

A 96 well plate is coated with cytokine (eg. interferon-(, IL-2)-specific antibody. Peripheral blood monocytes are then isolated from patient whole blood and are applied to the wells.

Antigen (ie. one of the peptides, fragments, derivatives or variants of the present invention) is added to stimulate specific T cells that may be present and the plates are incubated for 24 h. The antigen stimulates cytokine production which then binds to the specific antibody.

The plates are washed leaving a footprint where antigen-specific T cells were present.

A second antibody coupled with a suitable detection system, eg. enzyme, is then added and the number of spots are enumerated after the appropriate substrate has been added.

The number of spots, each corresponding to a single antigen-specific T cell, is related to the total number of cells originally added.

The above Example also describes use of an antigen that may be used to distinguish. TB infected individuals from BCG vaccinated individuals. This could be used in a more discriminative diagnostic assay.

The following Table 2 lists the preferred promoters, peptides, and corresponding DNA coding sequences of the present invention.

TABLE 2

| | amino acid | nucleic acid |
|---|---|---|
| name | sequence | sequence |
| Mce1 operon promoter | | 1 |
| Rv0167 | 2 | 3 |
| Rv0168 | 4 | 5 |
| Rv0169 | 6 | 7 |
| Rv0170 | 8 | 9 |
| Rv0171 | 10 | 11 |
| Rv0172 | 12 | 13 |
| Rv0173 | 14 | 15 |
| Rv0174 | 16 | 17 |
| Rv0175 | 117 | 118 |
| Mce2 operon promoter | | 18 |
| Rv0586 | 19 | 20 |
| Rv0587 | 21 | 22 |
| Rv0588 | 23 | 24 |
| Rv0589 | 25 | 26 |
| Rv0590 | 27 | 28 |
| Rv0591 | 29 | 30 |
| Rv0592 | 31 | 32 |
| Rv0593 | 33 | 34 |
| Rv0594 | 35 | 36 |
| Mce3 operon promoter | | 37 |
| Rv1964 | 38 | 39 |
| Rv1965 | 40 | 41 |
| Rv1966 | 42 | 43 |
| Rv1967 | 44 | 45 |
| Rv1968 | 46 | 47 |
| Rv1969 | 48 | 49 |
| Rv1970 | 50 | 51 |
| Rv1971 | 52 | 53 |
| Rv2462c promoter | | 54 |
| Rv2462c | 55 | 56 |
| SigE/htrA operon promoter | | 57 |
| Ry1221 | 58 | 59 |
| Rv1222 | 60 | 61 |
| Rv1223 | 62 | 63 |
| Rv1224 | 64 | 65 |
| Rv1477/1478 operon promoter | | 66 |
| Rv1477 | 67 | 68 |
| Rv1478 | 69 | 70 |
| Rv1919c promoter | | 71 |
| Rv1919c | 72 | 73 |
| Rv0384c promoter | | 74 |
| Rv0384c | 75 | 76 |
| Rv2671-2672-2673 promoter | | 77 |
| Rv2671 | 78 | 79 |
| Rv2672 | 80 | 81 |
| Rv2673 | 82 | 83 |
| Rv1909/1910/1912c promoter | | 84 |
| Rv1910c | 85 | 86 |
| Rv1911c | 87 | 88 |
| Rv1912c | 89 | 90 |
| Rv2461c/2460c promoter | | 91 |
| Rv2461c | 92 | 93 |
| Rv2460c | 94 | 95 |
| Rv1179c promoter | | 96 |
| Rv1179c | 97 | 98 |
| Rv1646 promoter | | 99 |
| Rv1646 | 100 | 101 |
| Rv2042c (Rv2043- | | 102 |
| 2037c) promoter | | |
| Rv2037c | 103 | 104 |
| Rv2038c | 105 | 106 |
| Rv2039c | 107 | 108 |
| Rv2040c | 109 | 110 |
| Rv2041c | 111 | 112 |
| Rv2042c | 113 | 114 |
| Rv2043c | 115 | 116 |

References:

1. McKinney, J. D., et al., Persistence of Mycobacterium tuberculosis in macrophages and mice requires the glyoxylate shunt enzyme isocitrate lyase [see comments]. Nature, 2000. 406(6797): p. 735-8.

2. Pelicic, V., et al., Efficient allelic exchange and transposon mutagenesis in Mycobacterium tuberculosis. Proc Natl Acad Sci USA, 1997. 94(20): p. 10955-60.

3. Lee, M. H., et al., Site-specific integration of mycobacteriophage L5: integration-proficient vectors for Mycobacterium smegmatis, Mycobacterium tuberculosis, and bacille Calmette-Guerin. Proc Natl Acad Sci USA, 1991. 88(8): p. 3111-5.

4. McShane, H., et al., Enhanced immunogenicity of CD4(+) t-cell responses and protective efficacy of a DNA-modified vaccinia virus Ankara prime-boost vaccination regimen for murine tuberculosis. Infect Immun, 2001. 69(2): p. 681-6.

5. Movahedzadeh, F., M. J. Colston, and E. O. Davis, Characterization of Mycobacterium tuberculosis LexA: recognition of a Cheo (Bacillus-type SOS) box. Microbiology, 1997. 143(Pt 3): p. 929-36.

Additional References:

Sambrook, J., E. F. Fritsch, and T. Maniatis. 1989. Molecular cloning: a laboratory manual, 2nd ed. Cold Spring Harbour Laboratory Press, Cold Spring Harbour, N.Y.

Lefever, P., O. Denis, L. De Wit, A. Tanghe, P. Vandenbussche, J. Content, and K. Huygen. 2000. Cloning of the gene encoding a 22-kilodalton cell surface antigen of Mycobacterium bovis BCG and analysis of its potential for DNA vaccination against tuberculosis. Infection and Immunity. 68:1040-1047.

Vordermeier, H. M., P. J. Cockle, A. O. Whelan, S. Rhodes, M. A. Chambers, D. Clifford, K. Huygen, R. Tascon, D. Lowrie, M. J. Colston, and R. G.

Hewinson. 2000. Effective DNA vaccination of cattle with the mycobacterial antigens MPB83 and MPB70 does not compromise the specificity of the comparative intradermal tuberculin skin test. Vaccine. 19:1246-1255.

Cheng, W., C. Hung, C. Chai, K. Hsu, L. He, C. Rice, M. Ling, and T. Wu. 2001. Enhancement of Sindbis virus self-replicating RNA vaccine potency by linkage of Mycobacterium tuberculosis heat shock protein 70 gene to an antigen. J. Immunol. 166:6218-6226.

Lalvani, A. et al., 2001. Enhanced contact tracing and spatial tracking of Mycobacterium tuberculosis infection by enumeration of antigen-specific T cells. The Lancet 357:2017-2021.

1. A method of identifying a mycobacterial nucleic acid promoter sequence which is induced or up-regulated during mycobacterial virulence, said method comprising:
infecting a macrophage target cell with a Mycobacterium tuberculosis host cell, which host cell contains a nucleic acid construct comprising a putative mycobacterial promoter sequence operably linked to a coding sequence of a reporter gene located down-stream from the promoter;
culturing the macrophage under conditions which support mycobacterial virulence; and
identifying a promoter sequence which is induced or up-regulated during virulence by detecting expression of the reporter sequence.

2. A method according to claim 1, wherein the induced or up-regulated promoter sequence is detected by increased expression of the reporter gene under conditions which support mycobacterial virulence when compared with the corresponding level of expression when cultured under conditions which do not promote mycobacterial virulence.

3. A method according to claim 1 or 2, wherein the putative promoter sequence is derived from M. tuberculosis or M. bovis.

4. A method according to any preceding claim, wherein the reporter sequence encodes a green fluorescence protein.

5. A method according to claim 4, wherein a mycobacterial host cell having a promoter induced or up-regulated during mycobacterial virulence is separated from other host cells by fluorescence activated cell sorting.

6. A method of identifying a mycobacterial gene the expression of which is induced or up-regulated during mycobacterial virulence, said method comprising:
identifying a mycobacterial promoter sequence which is induced or up-regulated during infection of a macrophage by a M. tuberculosis host cell, wherein the host cell contains a nucleic acid construct comprising said promoter sequence operably linked to a coding sequence of a reporter gene located down-stream from the promoter;
aligning by sequence homology the nucleic acid sequence of the promoter with published nucleic acid sequence data for the same mycobacterial species; and
identifying the associated nucleic acid coding sequence under the control of said promoter.

7. An isolated mycobacterial promoter obtainable by a method according to any of claims 1-5, wherein the promoter preferably has the nucleic acid sequence of SEQ ID NO: 1; 18; 37; 54; 57; 66; 71; 74; 77; 84; 91; 96; 99; or 102.

8. An isolated nucleic acid coding sequence obtainable by a method according to claim 6, wherein the coding sequence preferably has the nucleic acid sequence of SEQ ID NO: 3; 5; 7; 9; 11; 13; 15; 17; 20; 22; 24; 26; 28; 30; 32; 34; 36; 39; 41; 43; 45; 47; 49; 51; 53; 56; 59; 61; 63; 65; 68; 70; 73; 76; 79; 81; 83; 86; 88; 90; 93; 95; 98; 101; 104; 106; 108; 110; 112; 114; 116; or 118.

9. An isolated mycobacterial peptide or a fragment or derivative or variant thereof, wherein the peptide is encoded by a mycobacterial gene the expression of which gene is induced or up-regulated during infection of a macrophage by a M. tuberculosis host cell containing said gene.

10. A pharmaceutical composition comprising a peptide, or a fragment or variant or derivative thereof, wherein the peptide is selected from the group consisting of SEQ ID NO: 2; 4; 6; 8; 10; 12; 14; 16; 19; 21; 23; 25; 27; 29; 31; 33; 35; 38; 40; 42; 44; 46; 48; 50; 52; 55; 58; 60; 62; 64; 67; 69; 72; 75; 78; 80; 82; 85; 87; 89; 92; 94; 97; 100; 103; 105; 107; 109; 111; 113; 115; and 117.

11. An inhibitor of a mycobacterial peptide, wherein the peptide is encoded by a mycobacterial gene the expression of which gene is induced or up-regulated during infection of a macrophage by a M. tuberculosis host cell containing said gene, and wherein the inhibitor is capable of substantially preventing or inhibiting the mycobacterial peptide from exerting its native biological function or effect.

12. An inhibitor according to claim 11, selected from the group consisting of:
an antibiotic capable of targeting the induced or up-regulated mycobacterial gene, or the gene product thereof; and an antisense or triplex-forming nucleic acid sequence which is complementary to at least part of the inducible or up-regulatable gene.

13. An antibody which binds to a peptide encoded by a gene, or to a fragment or variant or derivative of said peptide, the expression of which gene is induced or up-regulated during infection of a macrophage by a M. tuberculosis host cell containing said gene.

14. An antibody according to claim 13, wherein the peptide is selected from the group consisting of SEQ ID NO: 2; 4; 6; 8; 10; 12; 14; 16; 19; 21; 23; 25; 27; 29; 31; 33; 35; 38; 40; 42; 44; 46; 48; 50; 52; 55; 58; 60; 62; 64; 67; 69; 72; 75; 78; 80; 82; 85; 87; 89; 92; 94; 97; 100; 103; 105; 107; 109; 111; 113; 115; and 117.

15. An attenuated mycobacterium in which a gene has been modified, the expression of which gene is induced or up-regulated during infection of a macrophage by a M. tuberculosis host cell containing said gene, thereby rendering the mycobacterium substantially non-pathogenic.

16. An attenuated mycobacterium according to claim 15, wherein the gene to be modified has a wild-type coding sequence corresponding to one of the group consisting of SEQ ID NO: 3; 5; 7; 9; 11; 13; 15; 17; 20; 22; 24; 26; 28; 30; 32; 34; 36; 39; 41; 43; 45; 47; 49; 51; 53; 56; 59; 61; 63; 65; 68; 70; 73; 76; 79; 81; 83; 86; 88; 90; 93; 95; 98; 101; 104; 106; 108; 110; 112; 114; 116; and 118.

17. An attenuated microbial carrier, comprising a peptide encoded by a gene, or a fragment or variant or derivative of said peptide, the expression of which gene is induced or up-regulated during infection of a macrophage by a M. tuberculosis host cell containing said gene.

18. An attenuated microbial carrier according to claim 17, wherein the peptide is selected from the group consisting of SEQ ID NO: 2; 4; 6; 8; 10; 12; 14; 16; 19; 21; 23; 25; 27; 29; 31; 33; 35; 38; 40; 42; 44; 46; 48; 50; 52; 55;. 58; 60; 62; 64; 67; 69; 72; 75; 78; 80; 82; 85; 87; 89; 92; 94; 97; 100; 103; 105; 107; 109; 111; 113; 115; and 117.

19. An attenuated microbial carrier according to claim 17 or 18, wherein the attenuated microbial carrier is attenuated salmonella, attenuated vaccinia virus, attenuated fowipox virus, or attenuated M. bovis (eg. BCG strain).

20. A DNA plasmid comprising a promoter, a polyadenylation signal, and a DNA sequence which corresponds to the coding sequence of a mycobacterial gene, or a fragment or variant or derivative of said DNA sequence, the expression of which gene is induced or up-regulated during infection of a macrophage by a M. tuberculosis host cell containing said gene, wherein the promoter and polyadenylation signal are operably linked to the DNA sequence.

21. A DNA plasmid according to claim 20, wherein the coding sequence of said gene is selected from the group consisting of SEQ ID NO: 3; 5; 7; 9; 11; 13; 15; 17; 20; 22; 24; 26; 28; 30; 32; 34; 36; 39; 41; 43; 45; 47; 49; 51; 53; 56; 59; 61; 63; 65; 68; 70; 73; 76; 79; 81; 83; 86; 88; 90; 93; 95; 98; 101; 104; 106; 108; 110; 112; 114; 116; and 118.

22. A DNA plasmid according to claim 20 or 21, wherein the promoter is selected from the group consisting of:
CMV; and
SV40 promoters;
and the polyadenylation signal is selected from the group consisting of:
SV40; and
bovine growth hormone polyadenylation signals.

23. An isolated RNA sequence which is encoded by the coding sequence of a mycobacterial gene, or a fragment or variant or derivative of said coding sequence, the expression of which gene is induced or up-regulated during infection of a macrophage by a M. tuberculosis host cell containing said gene.

24. An RNA vector comprising the RNA sequence of claim 23 and an integration site for a chromosome of a host cell.

25. Use of a peptide or fragment or variant or derivative according to claim 9 or claim 10, an inhibitor according to claim 11 or claim 12, an antibody according to claim 13 or claim 14, an attenuated mycobacterium according to claim 15 or claim 16, an attenuated microbial carrier according to any of claims 17-19, a DNA sequence corresponding to the coding sequence of a gene which is induced or up-regulated during infection of a macrophage by a M. tuberculosis host cell containing said gene, or a fragment or variant or derivative of said DNA sequence, a DNA plasmid according to any of claims 20-22, an RNA sequence according to claim 23, and/or an RNA vector according to claim 24, in the manufacture of a medicament for treating or preventing a mycobacterial infection.

26. A method of treating or preventing a mycobacterial infection, by administering to a patient a peptide or fragment or variant or derivative according to claim 9 or claim 10, an inhibitor according to claim 11 or claim 12, an antibody according to claim 13 or claim 14, an attenuated mycobacterium according to claim 15 or claim 16, an attenuated microbial carrier according to any of claims 17-19, a DNA sequence corresponding to the coding sequence of a gene which is induced or up-regulated during infection of a macrophage by a M. tuberculosis host cell containing said gene, or a fragment or variant or derivative of said DNA sequence, a DNA plasmid according to any of claims 20-22, an RNA sequence according to claim 23, and/or an RNA vector according to claim 24.

27. Use of a peptide or fragment or variant or derivative according to claim 9 or claim 10, or an antibody according to claim 13 or claim 14, or a polynucleotide probe comprising at least 8 nucleotides wherein said probe binds to at least part of a gene which is induced or up-regulated during infection of a macrophage by a M. tuberculosis host cell containing said gene, in the manufacture of a diagnostic reagent for identifying a mycobacterial infection.

28. A recombinant method of preparing a mycobacterial peptide, or a fragment or derivative or a variant of said peptide, wherein the peptide is encoded by a mycobacterial gene the expression of which is induced or up-regulated during infection of a macrophage by a M. tuberculosis host cell containing said gene, comprising expressing a nucleic acid sequence corresponding to the coding sequence of said gene, or a fragment or variant or derivative of said nucleic acid sequence, in a host cell.

29. An isolated peptide, an inhibitor, an antibody, an attenuated mycobacterium, an attenuated microbial carrier, an isolated RNA molecule, an RNA vector, or a DNA plasmid substantially as hereinbefore described with reference to the Examples.
